SEQUENCE LISTING (4 sequences)

SEQ ID NO: 1 - protein, 70 aa, Artificial Sequence
  The sequence is synthetized.
GPETLCGAELVDALQFVCGDRGFYFNKPTGYGSSSRRAPQTGIVDECCFRSCDLRRLEMYCAPLKPAKSA

SEQ ID NO: 2 - protein, 24 aa, Artificial Sequence
  The sequence is synthetized.
YQPPSTNKNTKSQRRKGSTFEEHK

SEQ ID NO: 3 - protein, 110 aa, Artificial Sequence
  The sequence is synthetized.
GPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEERK

SEQ ID NO: 4 - protein, 97 aa, Artificial Sequence
  The sequence is synthetized.
GPETLCGAELVDALQFVCGDRGFYFNKPTGYGSSSRRAPQTGIVDECCFRSCDLRRLEMYCAPLKPAKSAGGGYQPPSTNKNTKSQRRKGSTFEEHK

(19) United States
(12) Patent Application Publication
(10) Pub. No.: US 2023/0073755 A1
(43) Pub. Date: Mar. 9, 2023

(54) USE OF IGF1 IN COMBINATION WITH IGFEC24 IN PREPARATION OF MEDICINE FOR PROMOTING TISSUE REPAIR AND REGENERATION

(71) Applicant: Chongqing University, Chongqing (CN)

(72) Inventors: Liling TANG, Chongqing (CN); Xing WANG, Chongqing (CN); Huifang SUN, Chongqing (CN); Piaoyang LIU, Chongqing (CN); Xichao XU, Chongqing (CN); Ying CHEN, Chongqing (CN); Lu ZHANG, Chongqing (CN); Yuanyuan LIANG, Chongqing (CN)

(73) Assignee: Chongqing University, Chongqing (CN)

(21) Appl. No.: 17/793,052

(22) PCT Filed: Oct. 21, 2020
(86) PCT No.: PCT/CN2020/122442

§ 371 Date: Jul. 15, 2022

(30) Foreign Application Priority Data
Jan. 15, 2020 (CN) 202010041467.6

(51) Int. Cl. A61K 38/30; A61P 25/28

(52) U.S. Cl. CPC A61K 38/30; A61P 25/28

(57) ABSTRACT

A use of the combination of IGF1 and IGFEc24 in a preparation of a drug for promoting tissue repair and regeneration is provided. The amino acid sequence of the IGF1 is shown in SEQ ID NO: 1; the amino acid sequence of the IGFEc24 is shown in SEQ ID NO: 2; and the amino acid sequence of the IGF1-24 is shown in SEQ ID NO: 4.

### CROSS REFERENCE TO THE RELATED APPLICATIONS

This application is the national phase entry of International Application No. PCT/CN2020/122442, filed on Oct. 21, 2020, which is based upon and claims priority to Chinese Patent Application No. 202010041467.6, filed on Jan. 15, 2020, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBCQHT002 Sequence Listing.txt, created on Jul. 11, 2022, and is 3,430 bytes in size.

### TECHNICAL FIELD

The present invention relates to the field of biopharmaceuticals, and specifically to the application of IGF1 and IGF1Ec24 in the preparation of drugs that promote tissue repair and regeneration.

### BACKGROUND

The regenerative capacity of various tissues in lower animals is strong, while that of higher animals is weak. The proliferation of human terminal differentiated cells, including nerve, cardiac and skeletal muscle cells, disappears rapidly after birth. Once damaged, they become permanently absent. Therefore, when a person suffers from neurodegenerative diseases, myocardial infarction, myasthenia gravis, etc., The damaged cells cannot be repaired, resulting in permanent loss of that part of the organ. The damaged cells cannot be repaired, and the function of that part of the organ cannot be restored.

Neurodegenerative diseases are characterized by progressive loss of neuronal structure and function including Alzheimer's disease (AD), Parkinson's disease (PD), multiple sclerosis (MS), and amyotrophic lateral sclerosis (ALS). Current drug development for neurodegenerative diseases primarily targets specific gene mutations and protein products known to be associated with disease progression, with no curable drugs yet available.

Cardiovascular disease is the leading cause of death in the population worldwide. Myocardial infarction is defined as partial myocardial necrosis due to persistent and severe myocardial ischemia, followed by filling of necrotic myocardial cells by fibroblasts, formation of purpura tissue, and ventricular remodeling, which leads to a decrease in cardiac function and eventual induction of life-threatening heart failure. Current treatments for myocardial infarction include drug therapy, thrombolytic therapy, interventional therapy and surgical treatment. Although these therapies can improve ventricular remodeling and improve myocardial ischemic symptoms to a certain extent, none of them can repair the necrotic myocardial tissue and increase the number of cardiomyocytes, which cannot reverse the occurrence of eventual heart failure. Cell therapy, i.e. transplantation of exogenous or autologous cells to replace necrotic cardiomyocytes, is a promising treatment, but it is costly and complicated to operate, and cannot yet become a routine clinical treatment. Therefore, there is a lack of therapeutic agents that can treat myocardial infarction and restore cardiac function in clinical practice.

Therefore, the development of drugs that promote the proliferation of terminally differentiated cells is of clinical importance for the treatment of neurodegenerative diseases, myocardial infarction, and myasthenia gravis. In a previous study, recombinant protein IGF1-24 was found to have the ability to promote the proliferation of a variety of cells, and thus could be used for tissue repair and regeneration. Further studies revealed that the mechanism is the synergistic effect of two parts, carboxy-terminal sequence (same as insulin-like growth factor 1 sequence) and amino-terminal sequence (IGF1Ec24 peptide). However, whether the two individual proteins IGF1 and IGFEc24 are active in combination has not been reported.

### SUMMARY

In view of this, it is an object of the present invention to provide the application of IGF1 and IGFEc24 combination in the preparation of drugs for promoting tissue repair and regeneration, by combining insulin-like growth factor 1 and IGF1Ec24 peptide can promote the proliferation of a variety of terminally differentiated cells including cardiomyocytes, neurons, skeletal muscle cells, etc., with significantly better effect than IGF1 alone or IGF1Ec, which can be used to prepare drugs that promote tissue repair and regeneration.

Protocol I of the present invention, the combination of IGF1 and IGFEc24, or the use of IGF1-24 in the preparation of drugs that promote tissue repair and regeneration, the amino acid sequence of said IGF1 is shown in SEQ ID NO: 1; the amino acid sequence of said IGFEc24 is shown in SEQ ID NO: 2; the amino acid sequence of said IGF1-24 is shown in SEQ ID NO: 4.

Protocol II of the present invention, in the combination of IGF1 and IGFEc24, or IGF1-24 in the preparation of drugs for promoting tissue repair and regeneration, the amino acid sequence of said IGF1 is shown in SEQ ID NO: 1; the amino acid sequence of said IGFEc24 is shown in SEQ ID NO: 2; the amino acid sequence of said IGF1-24 is shown in SEQ ID NO: 4.

In the present invention, said IGF1 is administered at an amount of at least 90μg/kg; said IGF1Ec is administered at an amount of at least 104 μg/kg; said IGF1-24 is administered at an amount of at least 100 μg/kg.

Protocol III of the present invention, the combination of IGF1 and IGFEc24, or IGF1-24 in the preparation of drugs for the promotion of cardiomyocyte repair and regeneration, the amino acid sequence of said IGF1 is shown in SEQ ID NO: 1; the amino acid sequence of said IGFEc24 is shown in SEQ ID NO: 2; the amino acid sequence of said IGF1-24 is shown in SEQ ID NO: 4.

Preferably, said cardiomyocyte is a terminally differentiated cardiomyocyte. Protocol IV of the present invention, the use of IGF1 and IGFEc24 in combination, or IGF1-24 in the preparation of drugs for promoting nerve cell repair and regeneration, the amino acid sequence of said IGF1 is shown in SEQ ID NO: 1; the amino acid sequence of said IGFEc24 is shown in SEQ ID NO: 2; the amino acid sequence of said IGF1-24 is shown in SEQ ID NO: 4.

In the present invention, said neuronal cell is a terminally differentiated neuronal cell. Protocol V of the present invention, the combination of IGF1 and IGFEc24, or IGF1-24 in the preparation of drugs for the promotion of skeletal muscle cell repair and regeneration, the amino acid sequence of said IGF1 is shown in SEQ ID NO: 1; the amino acid sequence of said IGFEc24 is shown in SEQ ID NO: 2; the amino acid sequence of said IGF1-24 is shown in SEQ ID NO: 4 is shown.

In the present invention, said skeletal muscle cells are terminally differentiated skeletal muscle cells.

Protocol VI of the present invention, the combination of IGF1 and IGFEc24, or the use of IGF1-24 in the preparation of drugs for the treatment of neurodegenerative diseases, myocardial infarction or/and myasthenia, central nerve injury and peripheral nerve injury, the amino acid sequence of said IGF1 being as shown in SEQ ID NO: 1; the amino acid sequence of said IGFEc24 being as shown in SEQ ID NO: 2.

In the present invention, said neurodegenerative disease is Alzheimer's disease, Parkinson's disease.

### Advantageous Effects

The beneficial effect of the present invention is that the present invention discloses a new method of IGF1 and IGFEc24 combination, or IGF1-24 peptide combination for the preparation of tissue repair and regeneration drugs. The proliferation of cells is promoted by the action of the two proteins alone or in combination, so as to replenish tissue newborn cells and play a role in promoting tissue repair and tissue regeneration. It provides a new idea for the treatment of tissue repair and regeneration, and also lays the foundation for clinical treatment of neurodegenerative diseases, myocardial infarction, myasthenia gravis, central nerve injury and peripheral nerve injury, etc.

### BRIEF DESCRIPTION OF THE DRAWINGS

FIG. 1 shows the immunofluorescence observation of heart tissue of SD rats.

FIG. 2 shows the proliferation of cardiomyocytes in different experimental groups of SD rats detected by Edu.

FIG. 3 shows the proliferation of cardiomyocytes in C57 mice detected by Edu.

FIG. 4 shows the proliferation of neuronal cells in different experimental groups of SD rats detected by Edu.

FIG. 5 shows the immunofluorescence observation of hippocampal dentate gyrus tissue.

FIG. 6 shows the proliferation of immature neuronal cells in different experimental groups of C57 mice detected by Edu.

FIG. 7 shows the number of immature neuronal cells in different experimental groups of C57 mice.

FIG. 8 shows the proliferation of skeletal muscle cells in different experimental groups of SD rats.

FIG. 9 shows the HE staining of rat heart after isoproterenol injection.

FIG. 10 shows HE staining of rat heart after recombinant protein IGF1-24 treatment.

FIG. 11 shows Masson staining of rat heart after isoproterenol injection.

FIG. 12 shows Masson staining of rat heart after recombinant protein IGF1-24 treatment.

FIG. 13A-13C show the proliferation of rat cardiomyocytes after treatment with recombinant protein IGF1-24.

FIG. 14 shows the change of GAP-43 protein expression after sciatic nerve injury in rats.

FIG. 15 shows the immunofluorescence map and statistical graph of GAP43 in rat sciatic nerve.

FIG. 16 shows the changes of footprints and the detection of sciatic nerve index in rats before and after surgery.

FIG. 17 shows the changes of footprints and detection of sciatic nerve index in rats before and after treatment with recombinant protein IGF1-24.

### DETAILED DESCRIPTION OF THE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

### Example 1.Amino Acid Synthesis

In certain embodiments, IGF1, IGFEc24, IGF1Ec and IGF1-24 were synthesized, respectively. IGF1 is human insulin-like growth factor 1 with the amino acid sequence GPETLCGAELVDALQFVCGDRGFYFNKPTGYGSSSRRAPQTGIVDECCFRSCDLRRLEM YCAP LKPAKSA (SEQ ID NO: 1) is shown; IGFEc24 is a human insulin growth factor Ec24 peptide with the amino acid sequence YQPPSTNKNTKSQRRKGSTFEEHK (SEQ ID NO: 2) is shown; IGF1Ec consists of 110 amino acids, of which the amino-terminal 70 amino acid sequence is the same as IGF1 and the carboxy-terminal 24 amino acid sequence is the same as IGF1Ec24 peptide, and the amino acid sequence is GPETLCGAELVDALQFVCGDRGFYFNKPTGYGSSSRRAPQTGIVDECCFRSCDLRRLEM YCAPLKPAKSARSVRAQRHTDMPKTQKYQPPSTNKNTKSQRRKGSTFEERK (SEQ ID NO: 3) is shown; IGF1-24 consists of 97 amino acids, of which the sequence of 70 amino acids at the amino terminus is identical to IGF1, and the sequence of 24 amino acids at the carboxyl terminus is identical to IGFEc24 peptide, interspersed by three Gly linkers, the specific sequences are

(SEQ ID NO: 4)
GPETLCGAELVDALQFVCGDRGFYFNKPTGYGSSSRRAPQTGIVDECCFRS
CDLRRLEMYCAPLKPAKSAGGGYQPPSTNKNTKSQRRKGSTFEEHK.

### Example 2. Activity Test

In certain embodiments, adult female SD rats were divided into five groups: PBS group, IGF1 group, IGF1Ec group, IGF1-24 group, and IGF1+IGF1Ec24 peptide group, with three rats in each group. The rats in each group were distinguished by ear clipping for subsequent protein injection according to the different body weights of the rats.

The rats in each group were injected with PBS, IGF1, IGF1Ec24 peptide, IGF1Ec and IGF1-24 for 7 consecutive days. Recombinant protein IGF1-24 was injected at 100 μg/kg, and the injection of the remaining proteins in each group was converted according to the molar amount of IGF1-24 injection as follows: 90 μg/kg for IGF1, 8.9 μg/kg for IGF1Ec24 peptide, 104 μg/kg for IGF1Ec.

Before protein injection, the rats were first weighed with a bench scale, and their body weight and the amount of protein injected were recorded and converted; then the rats were fixed with a rat fixator and their tails were exposed, and their tails were soaked in hot water for 1-2 min to stretch the tail vessels and facilitate the search for blood vessels; then the tails were wiped with alcohol cotton balls; after the tail vein was identified, the needle was inserted into the vessel at a 45° angle to the tail vein and the drug was slowly pushed in; the needle before pressing the wound with cotton to stop the bleeding, and then pull out the needle.

After seven days of protein injection, the intraperitoneal injection of Edu solution at a concentration of 2.5 mg/kg was performed for seven days, so that the final total dose of each rat reached 50 mg/kg.

Female 13-month-old mice were divided into 2 groups, PBS group and IGF1-24 group, with 6 mice in each group, and the mice in each group were distinguished by ear tags for subsequent protein injection according to the different body weights of the mice. The mice in each group were injected with PBS and IGF1-24 for 7 consecutive days, and the recombinant protein IGF1-24 was injected at 100 μg/kg. After seven days of protein injection, Edu solution at a concentration of 2.5 mg/kg was injected intraperitoneally for seven consecutive days, so that the final total dose of each mouse reached 50 mg/kg.

(3) Cardiomyocyte proliferation effect assay
A. Heart collection and histological analysis.

After the rats were anesthetized with 10% chloral hydrate, the rats were fixed and the thoracic cavity was dissected with surgical equipment to obtain heart tissue; the heart tissue was washed 3 times with PBS to remove residual blood; the heart tissue was immersed in 4% paraformaldehyde and fixed overnight; the fixed heart tissue was removed and trimmed; the trimmed heart tissue was subjected to ethanol gradient dehydration: 60% ethanol for 2 h, 70% ethanol for 2 h, 90% ethanol 1.5 h, 95% ethanol 1.5 h, anhydrous ethanol 30min, anhydrous ethanol 30 min; the dehydrated heart tissues were transparent with xylene for 2 h; the transparent heart tissues were put into paraffin for embedding at 70° C. overnight; slices were made with a paraffin slicer with a thickness of 6 μm, fished after stretching in water at 40° C., and baked at 70° C. for 2 h to make heart tissue sections.
B. Immunofluorescence of heart tissue.

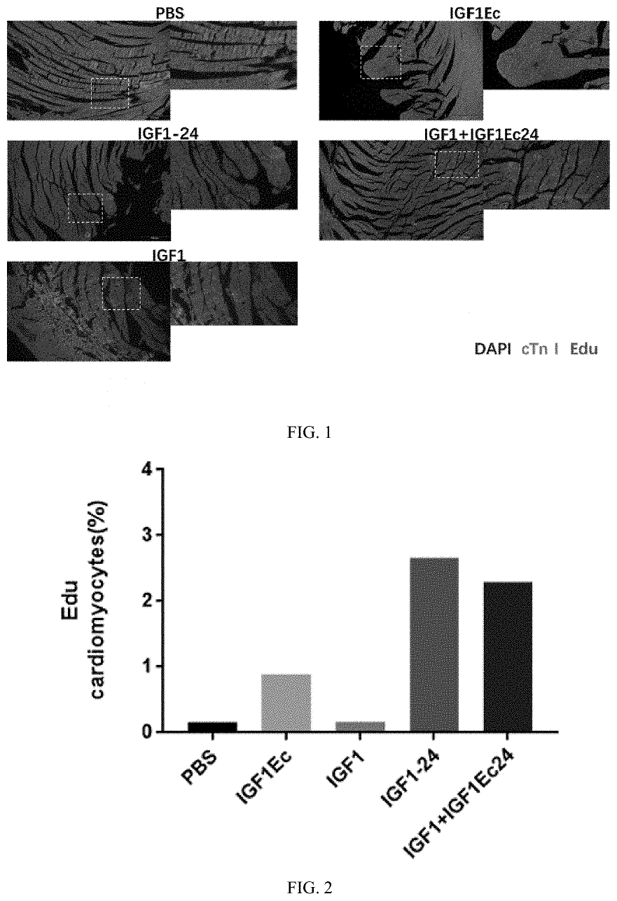

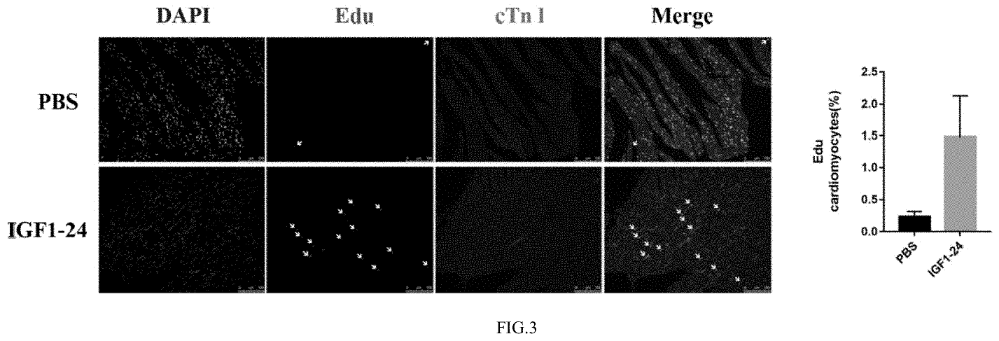

Sections were dewaxed by immersion in xylene for 20 min; then sections were hydrated: soak in ethanol twice, 5 minutes each time , 95% ethanol for 5 min, 75% ethanol for 5 min, 50% ethanol for 5 min, and tap water for 10 min; sections were immersed in a box containing sodium citrate antigen repair solution, and the box was heated in boiling water for 30 min, removed and cooled naturally to room temperature; sections were washed with PBS for 3 times for 10 min each; the sections were punched in 0.5% Triton for 30 min; washed with PBS for 3 times. 10 min each time; draw circles around the tissue with an immunohistochemistry pen and add 5% BSA dropwise to close for 1 h; aspirate 5% BSA and add cardiac troponin cTn I (1:500) dropwise and incubate overnight at 4° C.; wash 3 times with PBS for 10 min each time; incubate FITC secondary antibody (1:500) for lh away from light; wash 3 times with PBS for 10 min each time; add Edu reaction solution dropwise and incubated for 1 h with protection from light; washed 3 times with PBS for 10 min each time; incubated dropwise with DAPI for 10 min with protection from light; washed 3 times with PBS for 10 min each time; observed fluorescence under fluorescence microscope. The results are shown in FIGS. 1-3. The results showed that in adult rats, the number of Edu-positive cardiomyocytes reached 2.6% in the IGF1-24 group and 2.3% in the IGF1+IGF1Ec24 peptide group, and the effect of promoting cardiomyocyte proliferation was significantly higher than that in the PBS group, IGF1 group and IGF1Ec group. In aged mice, the number of Edu-positive cardiomyocytes reached 1.5% in the IGF1-24 group, compared with PBS group was 0.5%, and the recombinant protein IGF1-24 also reflected a higher effect of promoting cardiomyocyte proliferation in aged mice.

### (4) Detection of Neuroproliferation Effect

A. Brain tissue sampling and production of paraffin sections and frozen sections.

Adult female SD rats were anesthetized with 10% chloral hydrate, decapitated, brain was removed, skull was pried open with forceps to remove intact brain tissue, washed twice with PBS to remove residual blood, and fixed in 4% paraformaldehyde for 48 h; after fixation, brain tissue was removed, trimmed, and rinsed under running water overnight; the trimmed brain tissue was dehydrated in ethanol gradient: 60% ethanol for 3 h, 70% ethanol for 3 h, 80% ethanol for 3 h, 90% ethanol for 3 h, 95% ethanol for 1 h, anhydrous ethanol for 40 min, anhydrous ethanol for 40 min; the dehydrated brain tissue was cut into two pieces and transparent with xylene for 2-3 h; the wax was immersed overnight at 70° C. and embedded; the brain tissue was sliced 5 μm by paraffin slicer, dried after retrieving the slices and baked at 70° C. for lh to make brain tissue sections.

The 13-month-old C57 mice were anesthetized with 10% chloral hydrate, decapitated, and the brains were removed; the skulls were pried open with forceps to remove intact brain tissue, washed twice with PBS to remove residual blood, and fixed in 4% paraformaldehyde for 24 h; the tissue between the root of the optic nerve and the tegmentum was selected and rinsed in running water overnight; 30% sucrose was dehydrated, and after the tissue was sunk in sugar, 30% sucrose was replaced and sunk in sugar again; the optic nerve crossover was used as embedding surface, embedding in OCT. The tissues were snap frozen at −18° C. on a frozen sectioning machine, and after the snap freezing was completed, the tissue was sectioned at a thickness of 6 μm, and the sections were stored at −20° C.
B. Immunofluorescence of brain tissue sections.

Soak the sections in xylene to dewax for 20 min; hydration: anhydrous ethanol for a total of 2 times, 5 min/time, 95% ethanol for 5 min, 75% ethanol for 5 min, 50% ethanol for 5 min, tap water for 10 min; soak the sections in a box containing sodium citrate antigen repair solution, place the box in boiling water for 15 min, remove and cool naturally to room temperature; wash 3 times in PBS, 10 min/time; place the sections in 0.5% Triton for 30 min; wash 3 times with PBS for, 10 min/time; draw circles around the tissue with an immunohistochemistry pen and add 5% BSA dropwise to close for 1 h; aspirate 5% BSA and add double cortin DCX primary antibody (1:500) dropwise to incubate overnight at 4° C.; wash 3 times with PBS for 10 min/time; incubate FITC secondary antibody for 1 h; PBS wash 3 times for 10 min/time; incubate Edu dye for 1 h; PBS wash 3 times for 10 min/time; incubate DAPI for 10 min; PBS wash 3 times for 10 min/time; observe fluorescence under fluorescence microscope.

Remove frozen sections from −20° C. and restore to room temperature; wash 3 times 10 min/time with PBS; permeabilize sections in 0.5% Triton for 30 min; wash 3 times 10 min/time with PBS; draw circles around the tissue with an immunohistochemistry pen and add 5% BSA dropwise for 1 h; aspirate 5% BSA and incubate at 4° C. with double cortin DCX primary antibody (1:500) dropwise overnight; PBS wash 3 times for 10 min/time; incubate FITC secondary antibody for 1 h; PBS wash 3 times for 10 min/time; incubate Edu dye for 1 h; PBS wash 3 times for 10 min/time; incubate DAPI for 10 min; PBS wash 3 times for 10 min/time; observe fluorescence under fluorescence microscope.

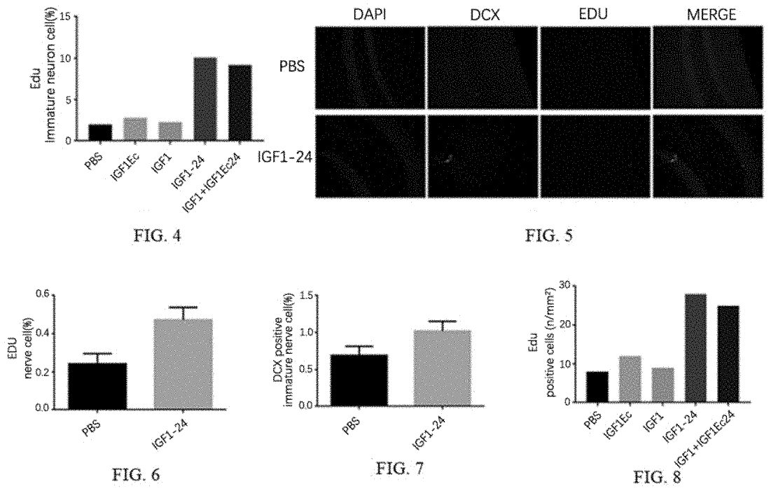

The microscope was selected with a 20× lens to observe the fluorescence staining of the dentate gyrus in the whole hippocampal region; the number of immature neurons and the number of newly proliferating neuronal cells in the dentate gyrus of the hippocampus were recorded. The results are shown in FIG. 4. In adult female SD rats, the proliferation of neuronal cells reached 10% in the IGF1-24 group and 8.9% in the IGF1+IGF1Ec24 peptide group, which promoted the proliferation of neuronal cells significantly more than PBS group, IGF1 group and IGF1Ec group. As results shown in FIG. 5 and FIG. 6, in the proliferation assay of 13-month-old C57 mice, the proliferation of neuronal cells in the PBS group was 0.24%, and the proliferation of neuronal cells in the IGF1-24 group was 0.47%, showing that IGF1-24 had a higher effect of promoting neuronal cell proliferation compared with the PBS group IGF1-24. As results shown in FIG. 7, in the neural differentiation assay of 13-month-old C57 mice, the immature neuronal cells in the PBS group were 0.68% and the immature neuronal cells in the IGF1-24 group were 1.01%, indicating that IGF1-24 had a higher effect of promoting neuronal cell differentiation than IGF1-24 in the PBS group.

### (3) Detection of Skeletal Muscle Cell Proliferation Effect

A. Gastrocnemius muscle tissue sampling and paraffin section production.

After the rats were anesthetized with 10% chloral hydrate, the gastrocnemius muscle tissues were stripped, washed 3 times with PBS, fixed in 4% paraformaldehyde and fixed at 4° C. for 48 h; after fixation, the gastrocnemius muscle tissues were removed and trimmed; the trimmed gastrocnemius muscle tissues were dehydrated with ethanol gradient: 70% ethanol for 20 min, 80% ethanol for 30 min, 90% ethanol for 40 min, 95% ethanol for 40 min, 95% ethanol for 40 min, anhydrous ethanol for 40 min, anhydrous ethanol for 40 min; the dehydrated gastrocnemius muscle tissue was transparent with xylene for 25 min; the wax was immersed overnight at 70° C. and embedded; paraffin slices were sliced 6μm, dried after retrieval and baked at 70° C. for 2 h to make gastrocnemius muscle tissue sections.
B. Immunofluorescence of gastrocnemius muscle tissue sections.

The sections were soaked in xylene for 20 min for dewaxing; hydration: anhydrous ethanol for a total of 2 times 5 min/time, 95% ethanol for 5 min, 75% ethanol for 5 min, 50% ethanol for 5 min, tap water for 10 min; the sections were soaked in a box containing sodium citrate antigen repair solution, the box was placed in boiling water for 15 min, removed and cooled naturally to room temperature; PBS washed 3 times 10 min/time; sections were punched in 0.5% Triton for 30 min; PBS washed 3 times for 10 min/time; immunohistochemistry pen was used to draw circles around the tissue and 5% BSA was added dropwise BSA closed for 1 h; aspirate 5% BSA, dropwise add junction protein Desmin primary antibody (1:500) incubated overnight at 4° C.; PBS washed 3 times 10 min/time; incubate FITC secondary antibody for 1 h avoiding light; PBS washed 3 times 10 min/time; incubate Edu dye for lh avoiding light; PBS washed 3 times for 10 min/time; fluorescence was observed under fluorescence microscope, and the results are shown in FIG. 8. The results showed that the number of Edu-positive skeletal muscle cells per square millimeter reached 28 in the IGF1-24 group and 23 in the IGF1+IGF1Ec24 peptide group, showing that the effect of IGF1-24 in promoting the proliferation of skeletal muscle cells was significantly higher than that of the PBS, IGF1 and IGF1Ec groups.

### Example 3: Validation of Myocardial Injury Repair

### (1) Construction and Treatment Of A Rat Model Of Myocardial Injury.

The rats were randomly selected and assigned into injection and control groups, with three rats in each group. The myocardial injury model was constructed by injecting an overdose of ISO at a dose of 80 mg/kg. The rats were fixed with a fixator to expose the abdomen; the rats were disinfected by wiping with alcohol cotton balls, and the amount of ISO injected was matched to their body weight, and then the rats were injected intraperitoneally for 1 week.

The rat was fixed with a fixator to keep its tail naturally down; before injection, the rat's tail was soaked in warm water for 3-4 min until the tail vein was seen clearly; then the rat's tail was wiped with an alcohol cotton ball for disinfection; then, when injecting the protein, the needle was injected at a minimal angle to the tail vein to keep it horizontal; after pushing the drug into the blood vessel, the wound was pressed with an alcohol cotton ball, and then the needle was withdrawn.

### (2) Detection of Cardiac Tissue Repair Effect

A. HE staining of cardiac tissue.

In order to fully remove the paraffin from the top of the slices, Paraffin sections of myocardial tissues were completely immersed in xylene twice, each time for 15 min; then the paraffin sections of cardiac tissues were placed into 100% ethanol solution, 95% ethanol solution, 75% ethanol solution, 50% ethanol solution and 3dH₂O in turn, with a soaking time of 10 min for each gradient. The paraffin sections were removed and hematoxylin was added dropwise at the myocardial tissue in the sections for nuclear staining with a staining time of 8 min. immediately after the staining was completed, the sections were rinsed with tap water for 5 min, then differentiation solution was added dropwise at the myocardial tissue in the sections with a differentiation time of 20 s, and finally the sections were rinsed with tap water for 5 min. the sections were removed and eosin staining solution was added dropwise at the tissue with a staining time of 1 min, and finally the sections were rinsed with tap water Rinse the slices for 5 min. soak the paraffin sections according to a gradient of 50%, 75%, 95% and 100% ethanol, each for 5 min; then soak them twice in xylene for 2 min/time. Paraffin sections were removed from xylene, keeping xylene still retained on the sections; neutral gum was quickly added dropwise, and coverslips were picked up with forceps, and the coverslips were put down slowly.

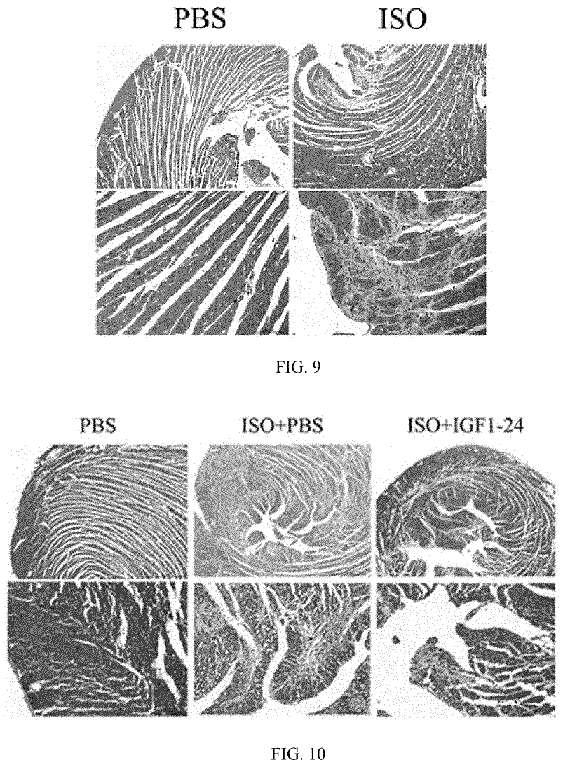

The results are shown in FIG. 9 and FIG. 10. From FIG. 9, it can be seen that the myocardial fibers in the PBS group were very neatly arranged both in the low magnification field of view and in the high magnification field of view; while the myocardial fibers in the ISO group were seen to be broken in the high magnification field of view and myocardial fiber lysis was observed, thus indicating that the injection of isoprenaline caused myocardial injury and the myocardial injury model was successfully constructed. As can be seen from FIG. 10, the PBS group was the uninjured group, in which myocardial tissue was neatly arranged, myocardial fibers were not broken, and no lesion sites were observed; the ISO+PBS group was the untreated group, in which myocardial tissue was severely damaged, with a large number of myocardial cells ruptured, nuclei exposed, myocardial fibers broken, and multiple lesion sites; the ISO+IGF1-24 group was the In the ISO+IGF1-24 group, a small amount of myocardial fibers were still broken and some nucleus were exposed, but the repair effect was obvious compared with the ISO+PBS group.
B. Masson staining of heart tissue.

The sections were soaked in xylene twice for 15 min/time; soaked sequentially according to a gradient of anhydrous ethanol, 95%, 75% and 50% alcohol solutions, each gradient for 5 min; finally the sections were soaked in triple distilled water for 5 min. equal amounts of Weigert iron hematoxylin A solution and B solution were mixed, and the mixture was added dropwise to the tissues, stained The sections were removed, and differentiation solution was added dropwise to the myocardial tissue for 20 s, and rinsed with tap water for 5 min. bluing solution was added dropwise to the tissue of the sections, and returned to blue treatment for 5 min, and rinsed with tap water for 5 min. lixin red magenta staining solution was added dropwise to the tissue of the sections, and staining time was 1 min, and rinsed with tap water for 5 min. 20% of Add phosphomolybdic acid solution dropwise on the tissue, stain for 5 min, pour off the phosphomolybdic acid solution on the section. Aniline blue staining solution was added dropwise for 5 min to re-stain the sections, and the aniline blue solution on the sections was poured off. The sections were soaked and washed with acetic acid solution for 10 min until no blue color came out of the sectioned tissue. The sections were soaked according to an alcohol gradient of 50%, 75%, 95% and anhydrous ethanol for 5 min/gradient; the sections were soaked twice with xylene for 2 min/gradient. The sections were removed and the xylene on the sections was kept in an incomplete evaporated state, neutral gum was added dropwise to the sections, and the coverslips were picked up with forceps to seal the sections.

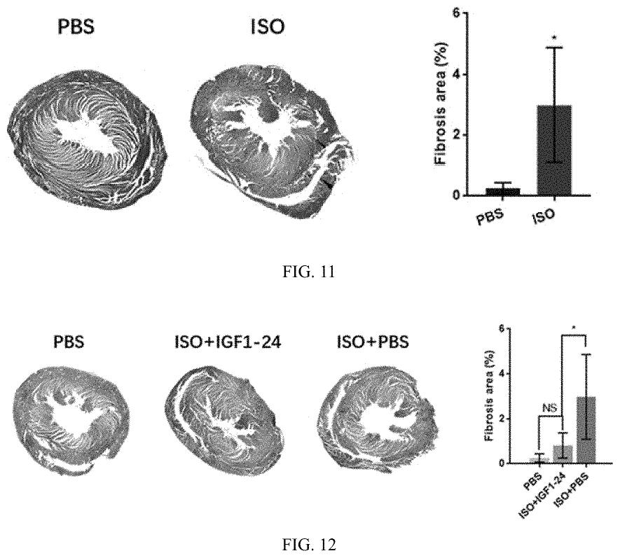

The results are shown in FIGS. 11-12. From FIG. 11, it can be seen that the area of blue collagen fibers in myocardial tissue was larger in the ISO-injured group than in the PBS-undamaged group; from the statistical results of the percentage of fibrotic area to myocardial tissue area, the difference between the ISO-injured group and the PBS-undamaged group was significant, indicating that the myocardial tissue of rats was damaged after the injection of high concentration of ISO, which led to the formation of fibrotic tissue. From FIG. 12, it can be seen that a large area of fibrotic tissue still existed in the ISO-injured group; the area of cardiac fibrosis in the ISO+IGF1-24-treated group was significantly reduced, and from the statistical results, there was no significant difference in the area of fibrosis in the treated group compared with the uninjured normal rats, which further indicates that the damaged myocardial tissue was somewhat restored after the recombinant protein IGF1-24 treatment.
C. Immunohistochemistry of heart tissues.

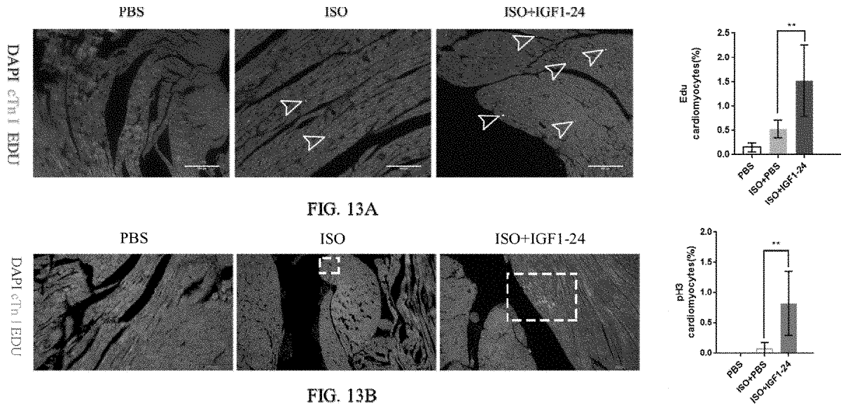

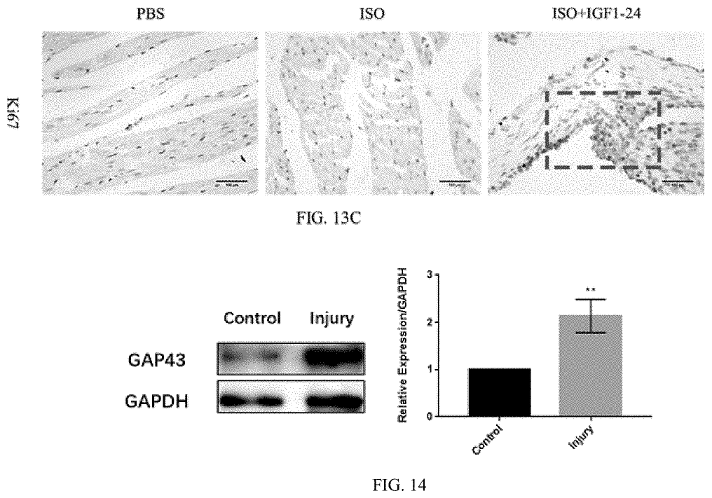

First, the heart sections were put into xylene and dewaxed twice for 10 min/time; then the paraffin sections of heart tissue were put into anhydrous ethanol, 95%, 75%, 50% alcohol solution and 3dH₂O with 10 min soaking time for each gradient; the sections were placed in the repair cassette, in which sodium citrate antigen repair solution was poured; then the repair cassette was put into a water bath with repair temperature of 100° C. for 30 min. The repair cassette was removed from the water bath and left to cool naturally at room temperature for 1-2 h. Finally, the tissue sections were washed three times with PBS for 5 min/time. The sections were removed, hydrogen peroxide was added dropwise to the tissue, incubated for 20 min, and then washed 3 times with PBS for 10 min/time. Serum closure solution was added dropwise on the sections and closed for 30 min, and the serum was decanted off after the time was up without washing the sections. Dilute the antibody with antibody diluent at a ratio of 1:500, add the diluent dropwise on the myocardial tissue, put it in the refrigerator at 4° C. and incubate it overnight. Paraffin sections of myocardial tissue were removed from the wet box and the primary antibody dilution was poured off. Wash the section with PBS 3 times for 10 min/time. Add biotin-labeled goat anti-rabbit IgG dropwise to the myocardial tissue. incubate for 30 min at room temperature and wash 3 times with PBS for 10 min/time. Horseradish enzyme-labeled streptavidin working solution was added dropwise to the myocardial tissue. Incubate for 15 min at room temperature and wash 3 times with PBS for 10 min/time. The DAB chromogenic solution was added dropwise on the myocardial tissue, and the staining time was determined according to the color change of the myocardial tissue under the microscope during staining, and immediately after the staining time, the staining was rinsed with tap water for 5 min. Hematoxylin was added dropwise on the myocardial tissue, and the time for re-staining was 5 min; then the differentiation solution was added dropwise on the myocardial tissue, and the differentiation time was 20 s; finally, the paraffin sections were rinsed with PBS and returned to blue. The sections were soaked according to a gradient of 50%, 75%, 95% and 100% ethanol for 10 min each, and finally the paraffin sections were soaked twice in xylene for 5 min each time. the paraffin sections were removed from the xylene, keeping the xylene still on the paraffin sections; neutral gum was quickly added dropwise, and the coverslips were picked up with forceps to seal the sections. The results were shown in FIG. 13C. The number of Ki67-positive cells in the treatment group differed significantly from that in the injury group, indicating that the recombinant protein IGF1-24 induced the proliferation of cardiomyocytes in the myocardial tissue after injury.
D. Immunofluorescence of cardiac tissue.

The specific procedure is described in Example 2. The results are shown in FIG. 13A and FIG. 13B. The results show that Edu and PH3 positive cardiomyocytes appeared in the post-injury myocardial tissue at a rate of 0.5% and 0.1%, respectively; the rates of Edu and PH3 positive cells in the myocardial tissue treated with recombinant protein IGF1-24 were 1.4% and 0.8%, respectively. This indicates that recombinant protein IGF1-24 induces proliferation of cardiomyocytes in myocardial tissues after injury.

### Example 4: Validation of Sciatic Nerve Injury Repair

### (1) Construction and Treatment of Rat Sciatic Nerve Injury Model

The 8-week-old female SD rats were randomly divided into sham-operated and operated groups, with 3 rats in each group. SD rats were anesthetized by intraperitoneal injection of 10% chloral hydrate, and after successful anesthesia, the rats were fixed, and their right hind limbs were exposed by shaving with a shaver, and disinfected twice with 75% alcohol. A 3-4 cm incision was made with a scalpel at the posterior lateral femur of the right hind limb to expose the biceps femoris muscle, and the muscle was bluntly separated with surgical scissors to expose the thick sciatic nerve. The sciatic nerve was clamped vertically with a microhemostatic forceps, buckled to the third buckle, maintained for 10 s, repeated 3 times at 5 s/interval, resulting in an injury area approximately 2 mm wide, and the sciatic nerve was put back in place and sutured layer by layer. Penicillin (40,000 units/100g) was injected intraperitoneally for three consecutive days to prevent wound infection.

On the constructed rat model of sciatic nerve injury, the recombinant protein IGF1-24 at a concentration of 100 μg/kg was then injected intraperitoneally for 14 consecutive days.

### (2) Sciatic Nerve Repair Effect Assay

A. GAP-43 Protein Assay
   \textcircled{1} Extraction of Total Protein from Sciatic Nerve

After obtaining the sciatic nerve, it was rinsed well in PBS, quickly wrapped in tin foil, placed in a grinding bowl and liquid nitrogen was added to it, and then quickly ground with a grinding pestle. The ground sciatic nerve powder was collected into EP tubes with a medicine spoon, and the sciatic nerve tissue was lysed by adding lysis solution and PMSF to the EP tubes. The EP tubes were placed on ice and shaken with an oscillator in every 5 min, repeated 8 times.
\textcircled{2} Using Western Blotting to Detect GAP-43 Expression

Equal amounts of total protein were mixed and dissolved in 4× SDS/PAGE sample buffer and heated at 100° C. for 10 minutes. The proteins were then separated on the gel and electrophoretically transferred to a PVDF membrane and milk closed for one hour. The membrane was incubated with primary antibody overnight at 4° C. On the next day, the membrane was washed three times with TBST, incubated with secondary antibody for one hour, and washed three more times with TBST. The developing solution was added to the membranes with the membranes face up and placed in the developing apparatus for development. The results are shown in FIG. 14. The results showed that the expression of GAP-43 protein in the sciatic nerve of rats in the surgical group was significantly higher than that in the sham-operated group. This indicates that the sciatic nerve of the rats was damaged.
B. Immunofluorescence of Sciatic Nerve

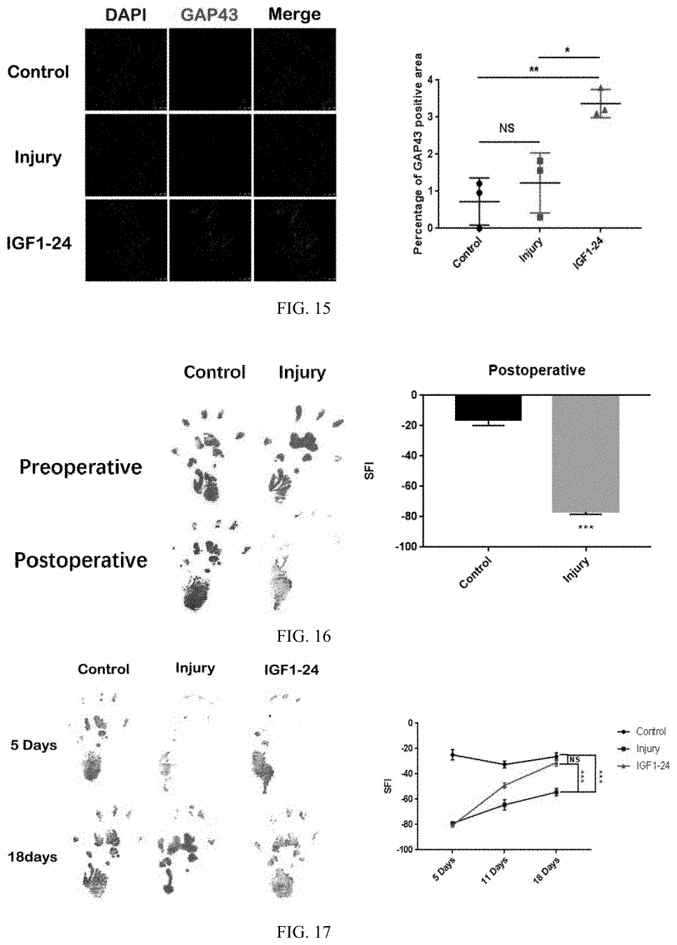

See Example 2 for the specific procedure, and the results are shown in FIG. 15. The results show that the expression of GAP-43 in the sciatic nerve of the treatment group (IGF1-24) remained at a high level after 14 days of recombinant protein IGF1-24 treatment. And the statistical results from immunofluorescence also show that the expression of GAP-43 in the treatment group was significantly different from that in the uninjured group (control) and the injury group (Injury).
C. Detection of Sciatic Nerve Function Index (SFI)

A dark box runway of 70 cm in length, 10 cm in width and 10 cm in height was made. White paper was laid in the runway and the hind limbs of the rats were fully stained with printing clay. The rats were put into the dark box runway and allowed to run through the dark box runway autonomously. The footprint length (PL), toe width (TS) and middle toe width (IT) were then measured and brought into the Bain formula for calculation.Bain formula: SFI=109.5 (ETS−NTS)/NTS−38.3 (EPL−NPL)/NPL+13.3(EIT−NIT)/NIT−8.8. The results are shown in FIGS. 16 and 17. As shown in FIG. 16, the footprints of the rats in the surgical group were blurred and no toes could be seen, while the footprints of the rats in the sham-operated group were clear and spreading. Calculation of the sciatic nerve index of the rats from the footprints revealed that the SFI of the rats in the sham-operated group was close to −20, while that of the rats in the operated group was close to −80. This indicates that the rat sciatic nerve injury model was successfully constructed. From the results in FIG. 17, it can be seen that although the sciatic nerve index of the injured group got increased, the difference between the injured group and the uninjured group was still significant. While the sciatic nerve index of the treated group was close to that of the uninjured group, indicating that the recombinant protein IGF1-24 played a repairing role for the damaged sciatic nerve.

The above described embodiments are only preferred embodiments to fully illustrate the present invention, and the scope of protection of the present invention is not limited thereto. The equivalent substitutions or transformations made by a person skilled in the art on the basis of the present invention are within the scope of protection of the present invention. The scope of protection of the present invention is governed by the claims.

What is claimed is:

1. A method of use of IGF1 and IGFEc24 in combination, or IGF1-24 in a preparation of drugs for promoting tissue repair and regeneration, wherein an amino acid sequence of the IGF1 is shown in SEQ ID NO: 1; an the amino acid sequence of the IGFEc24 is shown in SEQ ID NO: 2; an amino acid sequence of the IGF1-24 is shown in SEQ ID NO: 4.

2. The method according to claim 1, wherein the IGF1 is administered at an amount of at least 90 μg/kg; the IGFEc24 is administered at an amount of at least 104 μg/kg; and the IGF1-24 is administered at an amount of at least 100 μg/kg.

3. A method of use of IGF1 and IGFEc24 in combination, or IGF1-24 in a preparation of drugs for promoting cardiomyocyte repair and regeneration, wherein an amino acid sequence of the IGF1 is shown in SEQ ID NO: 1; an amino acid sequence of the IGFEc24 is shown in SEQ ID NO: 2, and an amino acid sequence of the IGF1-24 is shown in SEQID NO: 4.

4. The method according to claim 3, wherein a cardiomyocyte is a terminally differentiated cardiomyocyte.

5. A method of use of IGF1 and IGFEc24 in combination, or IGF1-24 in a preparation of drugs for promoting nerve cell repair and regeneration, wherein an amino acid sequence of the IGF1 is shown in SEQ ID NO: 1; an amino acid sequence of the IGFEc24 is shown in SEQ ID NO: 2; an amino acid sequence of said the IGF1-24 is shown in SEQ ID NO: 4.

6. The method according to claim 5, wherein a neuronal cell is a terminally differentiated neuronal cell.

7. A method of use of IGF1 and IGFEc24 in combination, or IGF1-24 in a preparation of a drug for promoting skeletal muscle cell repair and regeneration, wherein an amino acid sequence of the IGF1 is shown in SEQ ID NO: 1; an the amino acid sequence of the IGFEc24 is shown in SEQ ID NO: 2, and an amino acid sequence of the IGF1-24 is shown in SEQ ID NO: 4.

8. The method according to claim 7, wherein skeletal muscle cells are terminally differentiated skeletal muscle cells.

9. A method of use of IGF1 and IGFEc24 in combination, or IGF1-24 in a preparation of drugs for the a treatment of a neurodegenerative disease, myocardial infarction or/and myasthenia gravis, central nerve injury and peripheral nerve injury, wherein an amino acid sequence of the IGF1 is shown in SEQ ID NO: 1; an amino acid sequence of IGFEc24 is shown in SEQ ID NO: 2, and an amino acid sequence of the IGF1-24 is shown in SEQ ID NO: 4.

10. The method according to claim 9, wherein the neurodegenerative disease is Alzheimer's disease, Parkinson's disease.
